(19) United States
(12) Patent Application Publication
(10) Pub. No.: US 2017/0079364 A1
(43) Pub. Date: Mar. 23, 2017

(54) PROTECTIVE FACE SHIELD

(71) Applicant: PAULSON MANUFACTURING CORPORATION, Temecula, CA (US)

(72) Inventor: Roy PAULSON, Murietta, CA (US)

(73) Assignee: PAULSON MANUFACTURING CORPORATION, Temecula, CA (US)

(21) Appl. No.: 14/946,616

(22) Filed: Nov. 19, 2015

Related U.S. Application Data
(60) Provisional application No. 62/082,509, filed on Nov. 20, 2014.

(51) Int. Cl. A42B 3/22; A61F 9/06

(52) U.S. Cl. CPC A42B 3/225; A61F 9/065; A42B 3/222

(57) ABSTRACT

Apparatus and methods are disclosed for the rapid and reliable replacement of protective windows of the shields and electric-arc shields. A locking device releasably connects the face shield window with the helmet or cap bracket.

### CROSS REFERENCE TO RELATED APPLICATIONS

This application claims the benefit of U.S. Provisional Application Ser. No. 62/082,509, entitled “Protective Face Shield,” filed on 20 Nov. 2014, and is incorporated herein by reference in its entirety.

### FIELD OF INVENTION

The present apparatus relates generally to the field of safety devices which protect people from injury arising from electric-arc discharges and more particularly to protective helmets and face shields.

### BACKGROUND OF THE INVENTION

Electrical-arc flash hazards are a known threat in the workplace and must be addressed to protect people who may be exposed to this dangerous threat. Electric-arcs or flashes can result from short circuits developing from poor electrical grounding, failure of insulation, or workers inadvertently contacting exposed electrical circuit elements with objects such as tools. Electric-arcs have extremely high temperatures and the energy they radiate can result in serious or fatal injury. To protect workers from exposure to such electric-arcs, a number of protective safety devices have been developed. In particular, face shields employing transparent windows comprised of compositions which retain the ability of the user of the shield to clearly see the workspace and have the ability to substantially block harmful radiation are available. These devices are designed to provide protection against the thermal, optical, and mechanical hazards generated by arc flash events.

During the normal course of use of electric-arc shields, the transparent windows will often become scratched or cracked or otherwise degraded. Moreover, the U.S. Occupational Safety and Health Administration (OSHA) has established as a recommendation that electric-arc shield windows be replaced after two years of service. The protective window of such a protective shied is installed at the factory using appropriately designed and employed fixtures and tools to ensure that it is securely attached to the retainer structure of the shield and is properly registered with that structure so that maximum protection is provided to the user. In particular, the installation is designed to prevent radiation leaks around the periphery of the window.

However, in practice, it has generally not been feasible for a user of an electric arc shield to return the unit to a service facility for window replacement. Moreover, it has not been practical for users of electric-arc shields to be able to simply order replacement windows and install them into the shields themselves in a manner that would ensure that such as replacement process can be reliably undertaken by users in the field so as to guarantee a proper installation of the replacement window, that is, an installation which provides the same level of protection as the factory installed window and meet the established safety criteria.

Generally related prior art devices are shown in FIGS. 1-3. These are provided for reference purposes, to show examples of protective face shields.

### SUMMARY OF THE EMBODIMENTS OF THE INVENTION

Accordingly, it is a purpose of the present concept to provide an apparatus and method for replacing electric-arc protective windows in of variety of electric-arc shield assemblies which ensures that the new windows are properly installed so that the intended level of protection against electric-arcs is realized.

This concept provides a means by which electric-arc protective windows can be readily replaced at the worksite by the users of the electric-arc shield assemblies themselves without the need for calling in service personnel and without the significant loss of work time. The apparatus is configurable and adaptable to a wide variety of electric-arc shield structures and other face shields in general.

Other aspects of the invention will become apparent to those skilled in the art once the various embodiments have been shown and described. These aspects are not to be construed as limitations of the concept, but are merely to suggest some of the benefits that may be realized by the method and apparatus of the embodiments.

FIGS. 1-3 shows currently manufactured face shields that attach to hard hat helmets, at least one of these are attached by mounting to the accessory slots of those helmets. For example, FIG. 3 includes a transparent window comprised of a composition which absorbs a specified fraction of the harmful radiation produced by an electric-arc, while offering a wide viewing angle.

The embodiments which are disclosed herein are directed to a new and novel means of enabling the rapid and reliable replacement of the protective window in electric-arc shields similar to those described above as well as in other face and chin protective shields of suitable design. An approach in each embodiment of the present invention is to capitalize on the formable properties of the relatively rigid window material to provide what is tantamount to a spring-loaded window mounting. By employing mechanical structures including mounting posts and a quick-release capture mechanism, worn or damaged window can be deformed for release from its installed position and a replacement window can readily be sufficiently deformed for installation into the electric-arc shield structure in a secure, rapid process which locks it into the proper position. This entire replacement process can be accomplished in a matter of minutes, not only in a shop but also in the workplace, so work time lost in making the replacement is reduced to a minimum.

### BRIEF DESCRIPTION OF THE DRAWING

The purposes, features, and advantages of the disclosed device will be more readily perceived from the following detailed description, when read in conjunction with the accompanying drawing, wherein:

FIGS. 1-3 show currently available electric-arc shields;

FIG. 4 is an exploded perspective view of an embodiment of the invention;

FIGS. 5A-5H provide multiple views of the cap bracket of the face shield of FIG. 4;

FIGS. 6A-6D provide multiple views of the window retainer used with the face shield of FIG. 4;

FIG. 7 is a drawing of the window retainer rivet of the face shield of FIG. 4;

FIG. 8 is a plan front view of the window of the the shield of FIG. 4;

FIGS. 9A-9D provide multiple views of the window capture mechanism of the face shield of FIG. 4;

FIG. 10 is an exploded perspective view of an embodiment of the invention similar to that shown in FIG. 4 with the addition of a chin shield;

FIGS. 11A-11D are multiple views of a keyhole slot in the window of FIG. 8, the window retainer of FIG. 6, and the cooperation between the window retainer and the keyhole slot in the release and locked positions;

FIG. 12 is a sectional view of the window capture mechanism of FIG. 9 engaging the window in the cap bracket of FIG. 5; and

FIG. 13 is a perspective view of the protective shield of FIG. 10 in conjunction with a helmet.

### DETAILED DESCRIPTION OF THE PREFERRED EMBODIMENTS

As noted above, embodiments which are the subject of this invention are directed, in part, to a new and novel means of enabling the rapid and reliable replacement of protective face shield windows in the exemplary electric-arc shields described above as well as in other face shields of suitable design. An alternative embodiment also includes a replacement chin shield.

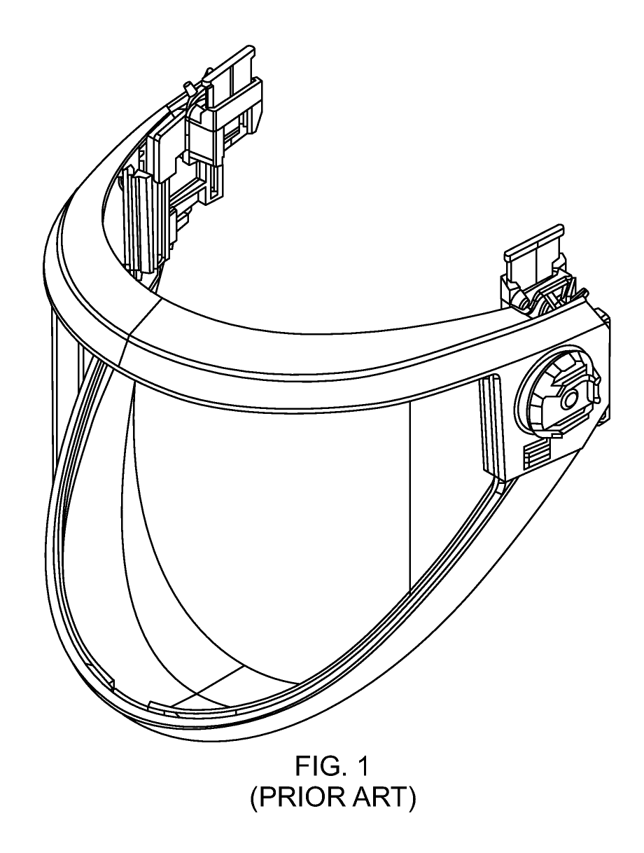

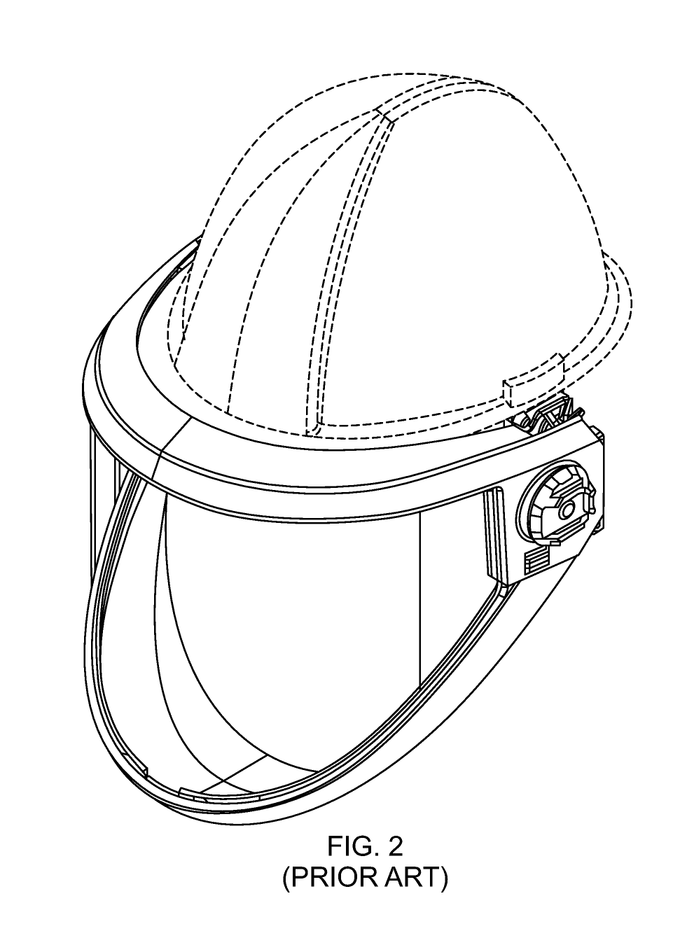

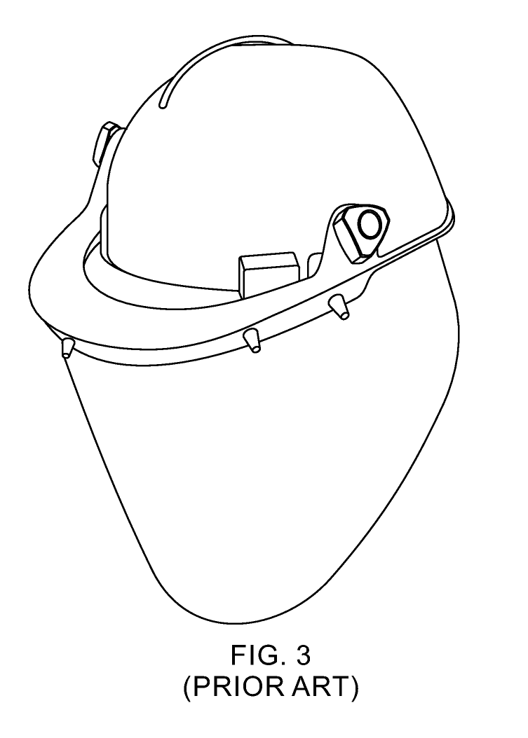

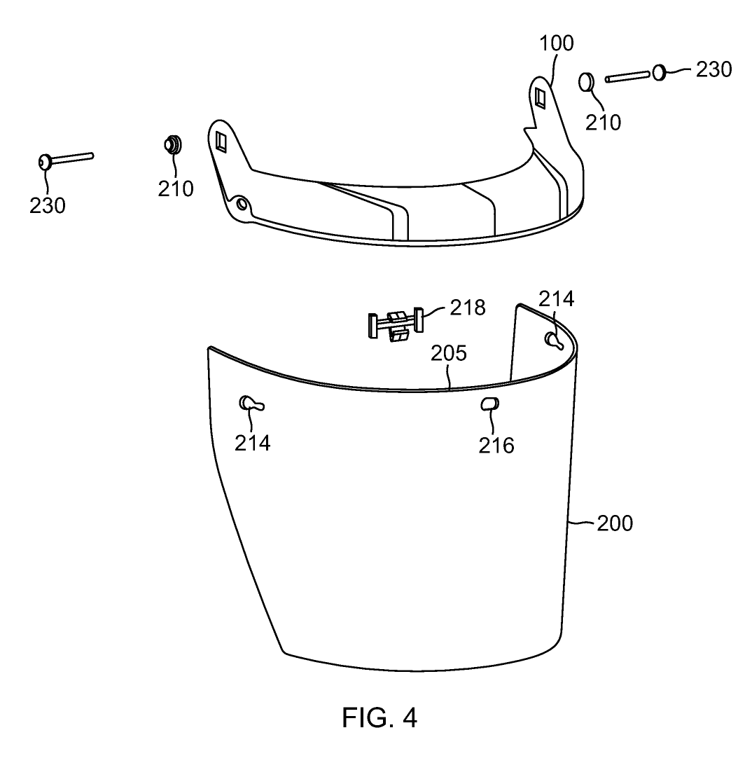

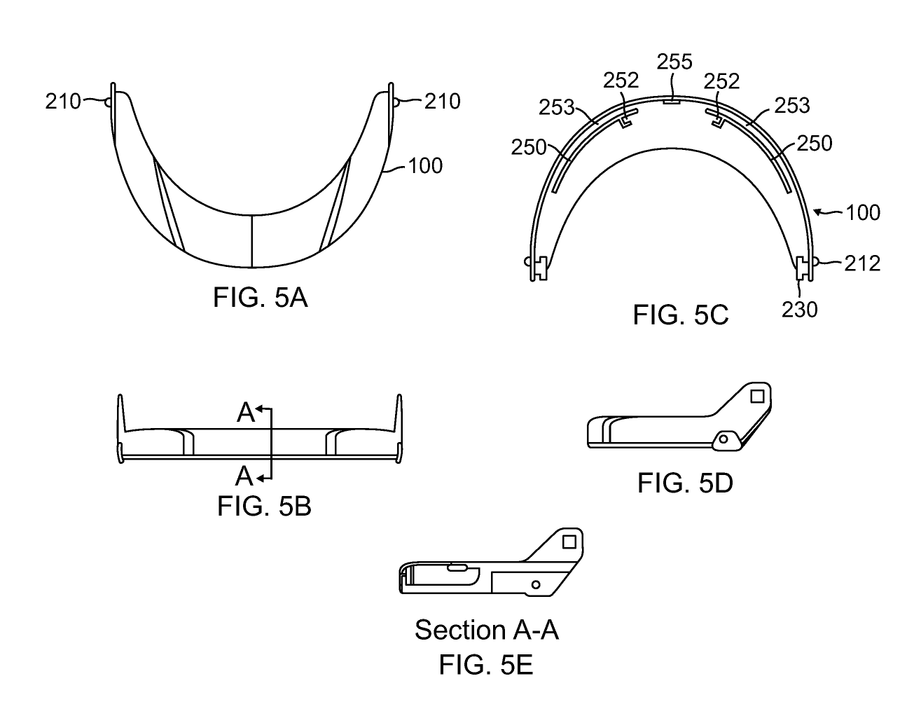

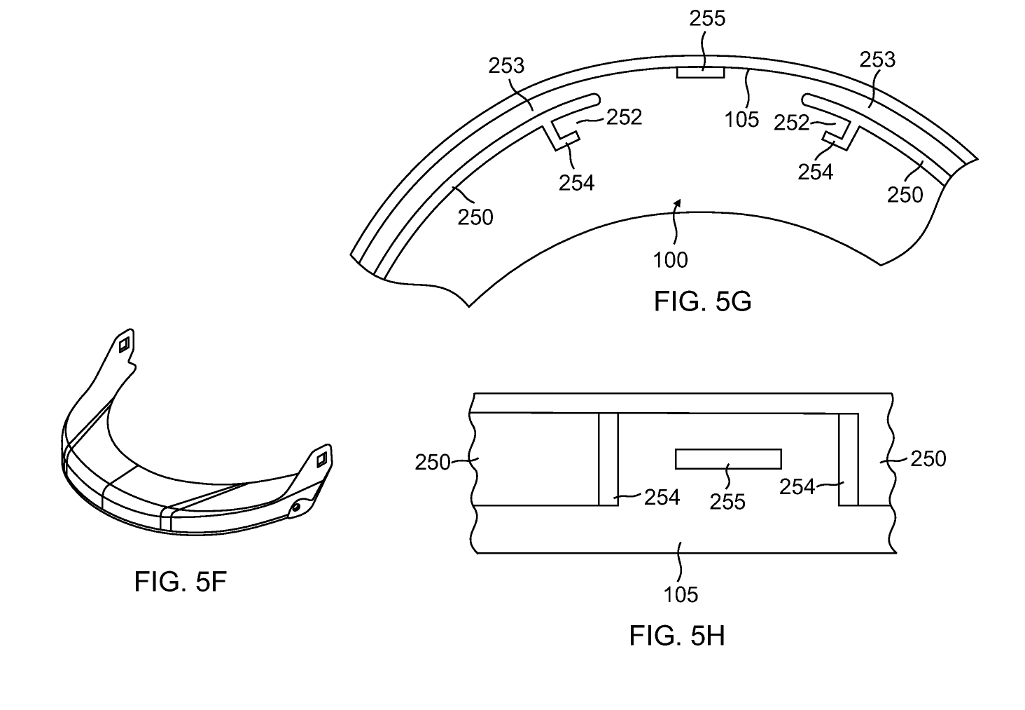

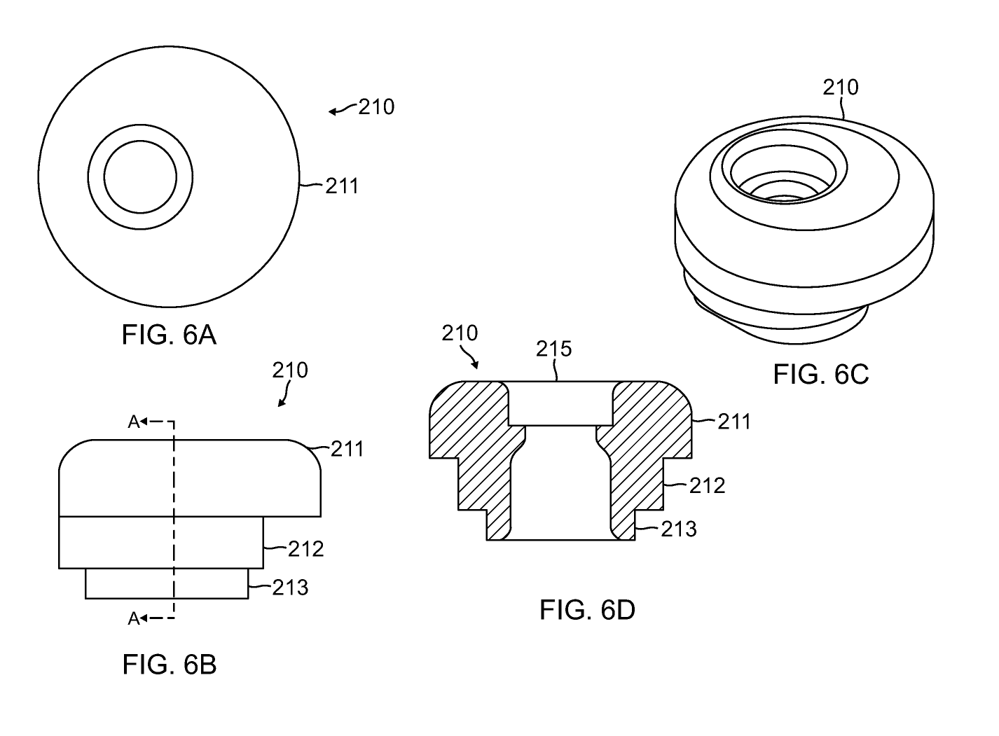

The preferred embodiment of the simplest form of the invention is shown in exploded view in FIG. 4. The cap bracket 100 can be attached to a helmet in a manner similar to the methods used to attach current electric-arc shields to helmets, as shown in FIGS. 1-3. However, in the present and preferred embodiment of a protective face shield, two window retainers 210 are installed on the opposite sides of the cap bracket temple areas using pop rivets 230, for example, in the locations 212 shown in FIG. 5. An exemplary embodiment of a window retainer which can be used with the FIG. 4 window is shown in FIG. 6.

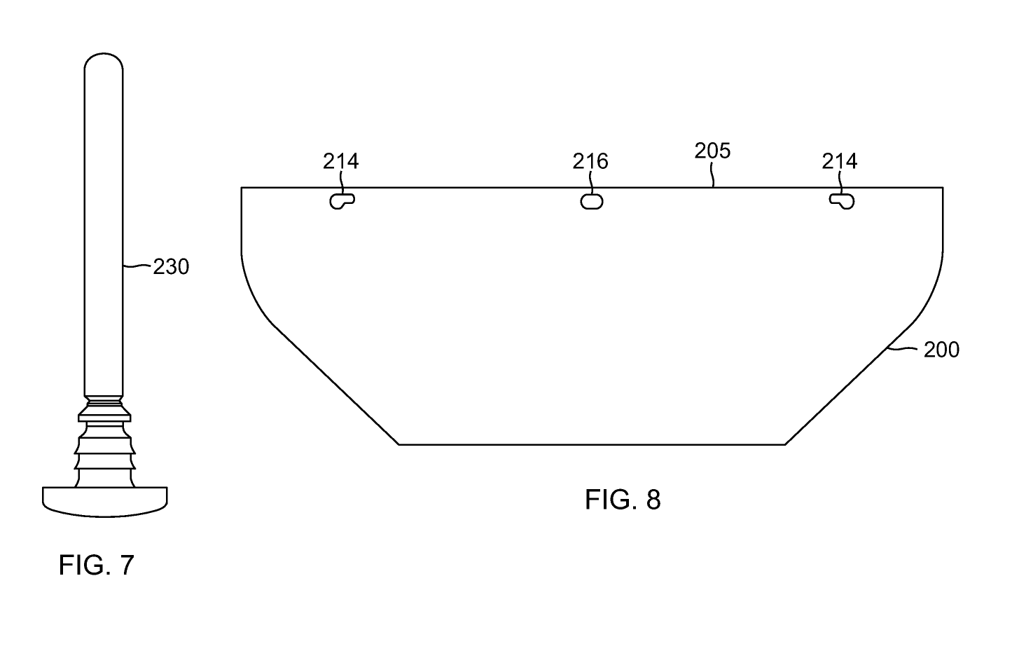

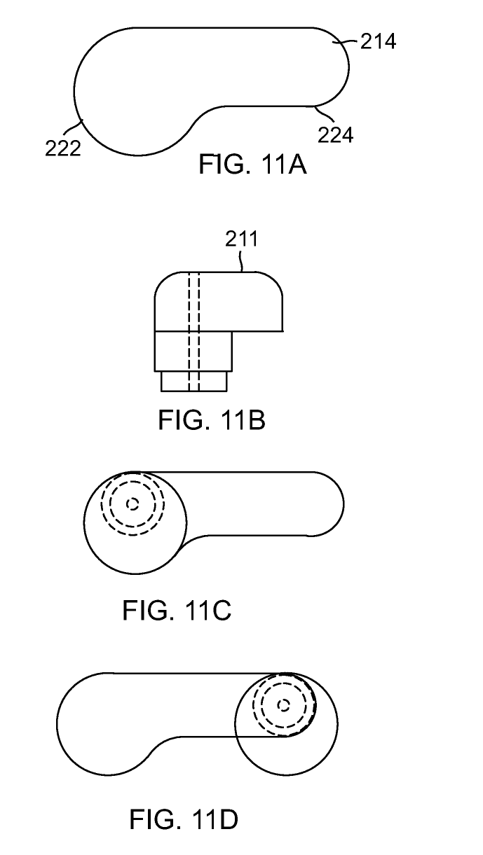

The window 200, shown in a flattened or Mercator-like projection in FIG. 8, is provided with two keyhole shaped slots 214 in two locations as shown, to match with the locations of the window retainers on the cap bracket. The precise location and orientation of each keyhole slot 214 is chosen so that when the window is attached to the cap bracket by means of the window retainers protruding through the smaller portion of the corresponding keyhole slots 214, the shape of the top portion of the window aligns with the shape of the lower rim of the cap bracket, as shown in FIG. 4. FIG. 11 indicates a preferred method of engagement of the keyhole slots 214 with the window retainers 210. In this method, the window retainers 210 (see FIG. 6) are formed so that cap portion 211 of each retainer is eccentric relative to cylinder portion 212 of the retainer. The cylinder portion is concentric with a spacer portion 213 of the retainer and with the axial passage 215 which is sized and shaped to accept and fasten the retainer to the cap bracket using pop rivet 230.

As shown in FIG. 11, each keyhole slot 214 is provided with a large diameter portion 223 and a smaller width or diameter portion 224. The large diameter portion is sized to allow clear passage of cap portion 211 of the window retainer. The small diameter portion is sized to allow passage of the cylinder portion of the window retainer but not the cap portion. To engage and release each side of the window from the cap bracket, the appropriate keyhole slot 214 is passed over the cap portion at its corresponding retainer and then moved so that the small portion of the keyhole slot engages the cylinder portion of the retainer.

The size and shape of the window are selected so that when the two keyhole slots on the window are fully engaged with the cylinder portions of the two window retainers mounted on the cap bracket, the window aligns with the cap bracket shape and the window material is in the arcuate state as shown in FIG. 4. To release the window from the cap bracket, the top central portion is rotated out of engagement with the cap bracket and then the slots 214 are easily slid on retainers 210 to disengage the window from the cap bracket. The window is then moved away from the cap bracket insofar as each keyhole slot will pass over the cap portion of the corresponding retainer. To replace the window with a replacement window, the above process is reversed, and then the window is rotated so that the top edge is engaged with the cap bracket, as will be described below.

A suitable material from which the window can be fabricated is a polycarbonate. This material can be impregnated with suitable dyes so that it absorbs a large fraction of the harmful radiation which may be produced during an electric arc. Moreover, this material has suitable properties which enable the deformations required to disengage and install a replacement window. Other materials which have the above characteristics could also be employed for the window.

The foregoing method and process describes how this embodiment of the invention enables installation of the upper two sides of the window of a face shield. However, a third point on the window must also be firmly and precisely attached to the cap bracket to provide an effective and positive three point support. A preferred location for such a third point of attachment is the upper central portion of the window. In order to enable rapid replacement of a damaged window, the method of attachment at this third point of attachment should be releasable with reasonable effort. The preferred method for connecting the window to the cap bracket at this central location in a manner which ensures a rapid, precise, firm, and releasable attachment employs a window capture mechanism or locking device 218, the location of which is shown in FIG. 4.

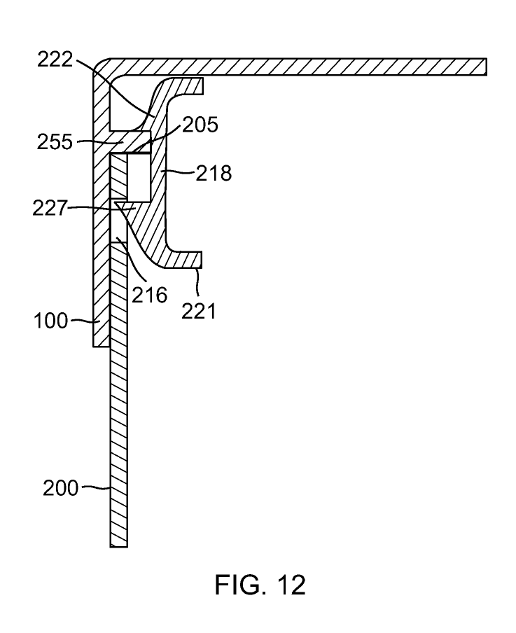

The capture mechanism 218 functions together with the structure shown in detail in FIGS 5C, 5G, and 5H, as well as with window 200 in FIG. 12. The underside of cap bracket 100 is shown, with baffles 250 formed in partial arcs on either side of bar 255 which projects inwardly from the front of the cap bracket. The facing spaced edges of the baffles are formed with grooves 252, with inside capture studs 254. Slots 253 between battles 250 and front rim 105 of the cap retainer are structured to receive top edge 205 of window 200.

From these detailed drawings it can easily be seen how window slots 214 (FIG. 4) can be engaged with retainers 210 while the top edge of the window is not engaged in slot 253 between baffles 250 and the inside front surface of the cap bracket. Once slots 214 are engaged, window 200 is rotated toward the cap bracket and it snaps into place (FIG. 12) when hook 227 enters hole 216 and top edge 205 of the window encounters bar 255.

To replace window 200, bar 221 is flexed slightly away from the window, releasing hook 227 from window hole 216. The window is then rotated out of slots 253, and then flexed slightly to disengage it from retainers 210.

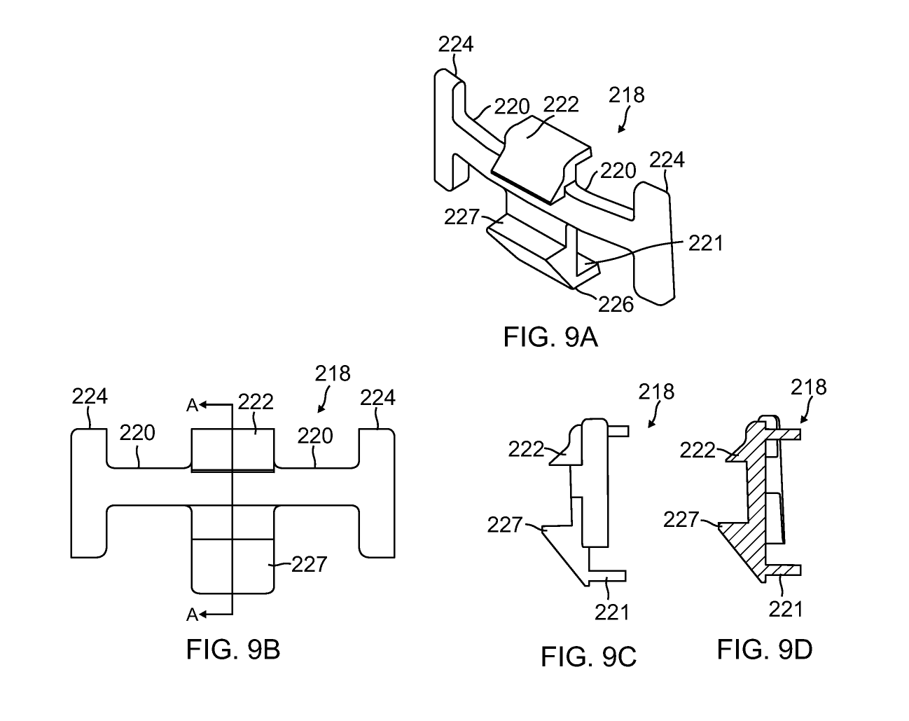

An embodiment of the window capture mechanism is shown in FIGS. 5, 9, and 12. This structure features two wings or mounting bars 224 and window engagement hook 227, with hook 227 connected to the mounting bars by means of torsion bars 220. This structure is preferably fabricated as a single piece from a suitable plastic or metal material which is relatively stiff and with flex properties which allow repeated deformations of the torsion bars without damage. One preferred material suitable for the fabrication of the window capture mechanism shown in FIG. 9 by a molding process is Ultramid A3W BK00464 Polyamide 66. Ultramid is a registered trademark of BASF Aktiengesellschaft

In the preferred embodiment, window capture mechanism 218 is mounted on the inside of cap bracket 100 with mounting bars 224 slid into opposing grooves 252 at the facing ends of baffles 250. When forced down into the grooves, hook 222 snaps over bar 255 for positive mounting on the cap bracket. Then the window can be removably engaged with the cap bracket as described above.

While the foregoing description discloses a particular embodiment, it should be clear that many variations of the specific embodiment described are also contemplated by the applicant. For example, the window retainers may be fabricated without an eccentric positioning of the retainer cap relative to the retainer cylinder and space. A structure comparable to the window capture mechanism shown and described above can be fabricated which attaches to a cap bracket-using adhesive or screw fasteners or rivets.

As an alternative means for rotatably coupling window 200 to cap bracket 100, the window can be equipped with retainer elements equivalent to window retainers 210, and the cap bracket can be formed with keyhole shaped slots equivalent to slots 214. The window would swivel with respect to the cap bracket in the same way as described above.

Additionally, slots 214 could be elongated rather than having a keyhole shape, as long as retainers have a cap or top T-shape that is larger than one end of the slot. Thus. the slot could have more of a teardrop or tapered shape.

Instead of hole 216 at the middle top edge of the window, a projection could be formed at that location. Hook 227 of capture mechanism 218 would then releasably engage that projection in the same manner that the hook releasably engages hole 216.

While the window capture mechanism described herein is preferably comprised of polyamide material, any suitable plastic or non-plastic material showing similar stiffness and flexing properties may be used. In essence, all embodiments of the invention as applied to an electric arc-shield with a single face shield window capitalize on the flexible properties of any window comprised of a material which enable it to be deformed from its resting shape to the degree necessary to enable a position in the top center of the window to engage a structure similar to the window capture mechanism structure as shown in FIG. 12, white the two upper corners of the window are held in a fixed positions to a cap bracket similar to that in FIG. 5. The means of fixing the upper corners of the window may involve structures similar to the keyhole and window retainer as in FIGS. 4 and 6 or any other structure which would perform the same function, that is, detachably fixing the upper outer portions of an electric-arc shield window to fixed positions on a cap bracket. A different design detail for a window capture mechanism may also be used provided it performs the same “snap into place” function as the window capture mechanism described above.

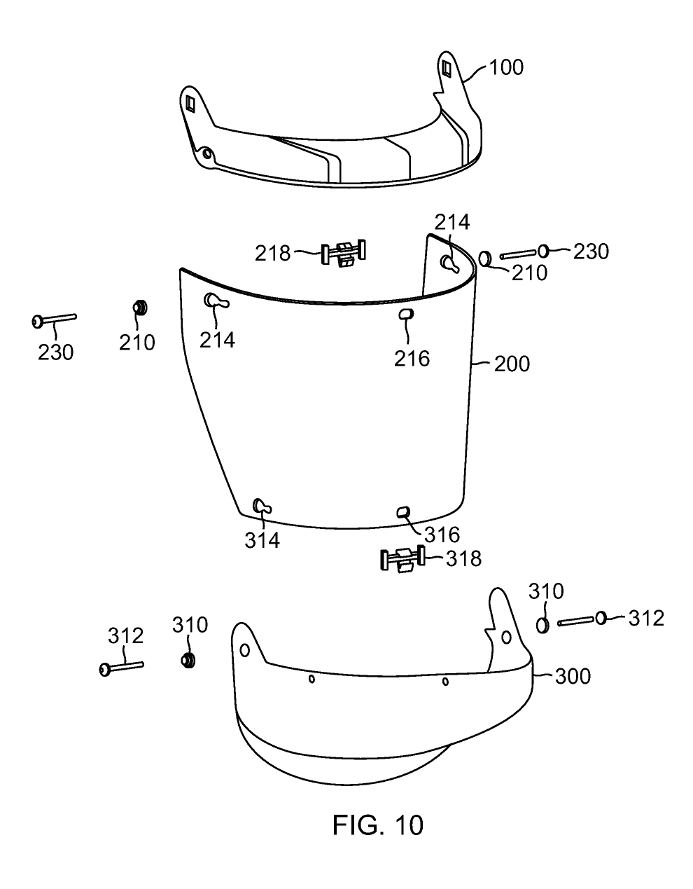

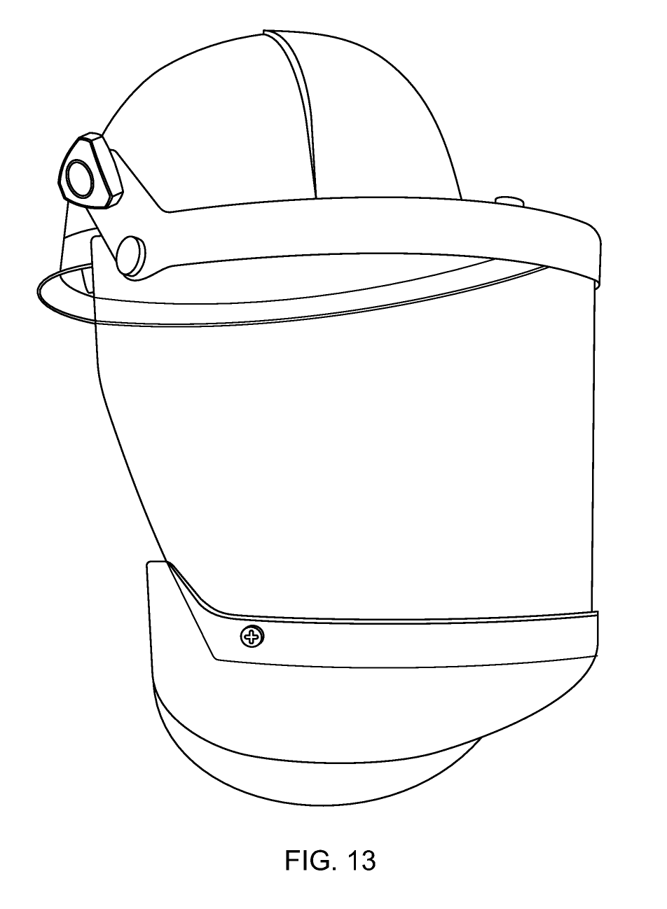

The embodiment of the invention described above was applied to an electric-arc shield with it single window face shield. However, electric-arc shields are often provided with the addition of a separate chin shield, with the chin shield being attached to the lower edge of the face shield, as shown in FIGS. 10 and 13. In some cases, the chin shield is attached to the two lateral lower edges of the face shield using rivets or other pivots, enabling the chin shield to be rotated away from the chin of the user. In other cases, the chin shield may be rigidly attached to the face shield using screws of an adhesive. In either case, the embodiments of the invention presented above will continue to function, enabling the face shield to be easily removed in the workplace if needed for repair or replacement. But in this case, the entire assembly of face shield with attached chin shield would be replaced.

An alternative embodiment of the current invention enables the rapid removal and replacement of the chin protector or shield alone should it become damaged, without the use of tooling. The same principles of the use of keyhole slots in the face shield window and a window capture mechanism are applied to the lower edge of the face shield to effect this embodiment, as shown in FIGS. 5, 10, and 12. Keyhole shaped slots 314 are provide on each lower side of the face shield window. Hole 316 is provided in the center and close to the lower edge of the face shield window. Window retainers 310 of a design similar to that of the window retainers 210 installed onto the cap bracket are installed on either side of the chin shield with pop rivets 312, as shown in FIG. 10. A window capture mechanism 310 similar or identical to that used on the cap bracket is attached to the center of the chin shield. This attachment may be by means of slots built into the chin shield similar to the grooves 252 built into the cap bracket for this purpose, where the chin protector is also formed with baffles to create slots equivalent to slots 253 in the cap bracket. Alternatively, the capture mechanism can be attached to the chin shield using an adhesive or using screws or nuts and bolts.

The chin shield is then attached to the face shield window by engaging the two window retainers 310 with the corresponding keyhole slots 314 in window 200, with the chin shield positioned away from the lower edge of the face shield window. Once the chin shield is attached to the face shield window by means of the two window retainers, the chin shield is rotated upward to engage the capture mechanism and snap into place, as described above with the top edge of the window. The same method for the release and removal of the face shield window from the cap bracket as described above can be applied here for the removal of the face shield window from the chin shield.

This embodiment of the invention thus enables the rapid removal and replacement of the chin shield with or without the replacement of the face shield window.

While particular embodiments of the present invention have been disclosed, it is to be understood that various different modifications and combinations are possible and are contemplated within the true spirit and scope of the disclosed embodiments. There is no intention, therefore, of limitations to the exact disclosures herein presented.

What is claimed is:

1. A protective face shield apparatus for use with a safety helmet configured with a mechanism for receiving the face shield apparatus, the face shield apparatus comprising:
a cap bracket configured with a device to engage the helmet mechanism, said cap bracket having extremities at opposite temple areas of the safety helmet;
a face protective window formed with a top edge defining upper corners of the window, and a bottom edge;
releasable pin and locating hole arrangements at the extremities of said cap bracket and adjacent to the top corners of said window by which said window is releasably and swivalably engageable with said cap bracket; and
a capture mechanism in a central area of said cap bracket for releasably engaging the bottom edge of said window.

2. The apparatus of claim 1, wherein:
said window is formed with a hole in the lop edge; and
said capture mechanism is formed with a hook capable of being flexed to disengage the top edge hole in said window.

3. The apparatus of claim 1, wherein said window is comprised of semi-transparent material.

4. The apparatus of claim 1, wherein said window capture mechanism is comprised of material with sufficient deformability and geometry to capture and release said window by deformation of said capture mechanism.

5. The apparatus of claim 1, wherein said releasable arrangements for swivelably engaging said window and cap bracket comprise:
a window retainer pin mounted in each cap bracket extremity; and
a locating hole formed at each top corner of said window, each hole being configured to releasably engage a said retainer pin.

6. The apparatus of claim 1, wherein said locating holes have a keyhole shape.

7. The apparatus of claim 1, and further comprising:
a chin shield having an upper edge with at least two attachment devices;
said window being configured with at least two attachment elements at its bottom edge;
whereby said chin shield is mounted to said window by respective engagement of said at least two attachment devices and said two attachment elements.

8. The apparatus of claim 7, wherein the chin shield attachment devices and the window attachment elements comprise releaseable pin and locating hole arrangements by which said chin shield is releasably and swivalably engageable with said window.

9. The apparatus of claim 8, and further comprising a chin shield capture mechanism mounted to said window, said chin shield being releasably engageable by said chin shield capture mechanism.

10. The apparatus of claim 7, wherein said chin shield is comprised of semi-transparent material.

11. A replaceable protective face shield for a face shield apparatus for use with a safety helmet, the helmet having a cap bracket having extremities at opposite temple areas of the helmet, the face shield comprising:
a face protective window formed with a top edge defining upper corners of the window, and a bottom edge;
releasable pin and locating hole arrangements at the extremities of said cap bracket and adjacent to the upper corners of said window by which said window is releasable and swivalably engageable with said cap bracket; and
a capture mechanism in a central area of said cap bracket for releasably engaging the bottom edge of said window.

12. The face shield of claim 11, wherein:
said window is formed with a hole in the top edge; and
said capture mechanism is formed with a hook capable of being flexed to disengage the top edge hole in said window.

13. The face shield of claim 11, wherein said window capture mechanism is comprised of material with sufficient deformability and geometry to capture and release said window by deformation of said capture mechanism.

14. The face shield of claim 11, wherein said releasable arrangements for swivelably engaging said window and cap bracket comprise:
a window retainer pin mounted in each cap bracket extremity; and
a locating hole formed at each top corner of said window, each hole being configured to releasably engage a said retainer pin.

15. The face shield of claim 11, wherein said locating holes have a keyhole shape.

16. A protective face shield apparatus for use with a safety helmet, the face shield apparatus comprising:
a cap bracket having means for attachment to the safety helmet, said cap bracket having extremities at opposite temple areas of the safety helmet;
at least two window retainers at said cap bracket extremities;
a capture mechanism in a central area of said cap bracket; and
a face protective window formed with locating holes at extremeties of said window, said locating holes being configured to removably engage said window retainers of said cap bracket, said window also being configured to be removably engaged by said capture mechanism, said window further having deformability enabling sufficient flexing of said window to selectively enrage and disengage said window retainers and said capture mechanism.

17. The apparatus of claim 16, wherein said window is comprised of semi-transparent material impregnated with dyes selected to absorb radiation produced by an electric-arc flash.

18. The apparatus of claim 16, wherein said window capture mechanism releasably locks the window to said cap bracket.

19. The apparatus of claim 18, wherein said window capture mechanism is rigidly affixed to said cap bracket.

20. The apparatus of claim 16, wherein said locating holes have a keyhole shape.

21. The apparatus of claim 10, wherein the cap bracket is configured to enable the window to be rotated about an axis connecting said window retainers.

22. The apparatus of claim 19, wherein said window capture mechanism is comprised of material with sufficient deformability and geometry to capture and release said window by deformation of said capture mechanism.

23. The apparatus of claim 16, and further comprising:
at least two chin shield locating pins and a chin shield capture mechanism on said window; and
a chin shield, said chin shield being formed with locating holes at extremeties of said chin shield and configured to engage said chin shield locating pins, said chin shield also being configured to be engaged by said chin shield capture mechanism.

24. The apparatus of claim 23, wherein said chin shield further has deformability enabling sufficient flexing to engage and disengage said chin shield locating pins and said chin shield capture mechanism.

25. The apparatus of claim 23, wherein said chin shield is comprised of semi-transparent material impregnated with dyes selected to absorb radiation produced by electric-arc flash.

26. The apparatus of claim 23, wherein said chin shield capture mechanism is comprised of material with sufficient deformability and geometry to capture and release said chin shield by deformation of said chin shield capture mechanism.

27. The apparatus of claim 23, wherein said chin shield capture mechanism is rigidly affixed to said chin shield.

28. The apparatus of claim 23, wherein said chin shield capture mechanism releasably locks said window to said chin shield.
